(19) United States
(12) Patent Application Publication
(10) Pub. No.: US 2015/0374394 A1
(43) Pub. Date: Dec. 31, 2015

(54) SONOLYSIS WITH BIODEGRADABLE NANOPARTICLES

(71) Applicant: THE BOARD OF TRUSTEES OF THE UNIVERSITY OF ARKANSAS, Little Rock, AR (US)

(72) Inventors: Michael J. BORRELLI, Little Rock, AR (US); Ajay P. MALSHE, Little Rock, AR (US); Eric HAMILTON, Little Rock, AR (US); Kaleb SMITHSON, Little Rock, AR (US); Dheeraj AHLUWALIA, Little Rock, AR (US)

(73) Assignee: THE BOARD OF TRUSTEES OF THE UNIVERSITY OF ARKANSAS, Little Rock, AR (US)

(21) Appl. No.: 14/429,848

(22) PCT Filed: Sep. 24, 2013
(86) PCT No.: PCT/US2013/061396

§ 371 Date: Mar. 20, 2015

Related U.S. Application Data
(60) Provisional application No. 61/705,429, filed on Sep. 25, 2012.

(51) Int. Cl. A61B 17/22; A61M 5/14; A61M 37/00; A61K 9/14; A61K 41/00

(52) U.S. Cl. CPC A61B 17/22004; A61K 9/146; A61K 41/0047; A61M 37/0092; A61M 5/14; A61B 2017/22005; A61B 2017/22088

(57) ABSTRACT

The present invention provides methods for lysing a thrombus or ablating a mass in a subject by administering a plurality of biodegradable nanoparticles and delivering ultrasound to the subject. Also provided is a method for delivering a therapeutic agent to a tumor in a subject by administering a plurality of biodegradable nanoparticles comprising the therapeutic agent and delivering ultrasound to the subject.

### FIELD OF THE INVENTION

The present invention relates to the use of ultrasound and biodegradable nanoparticles to lysis thrombi or other masses, and/or deliver therapeutic agents to tumors.

### BACKGROUND OF THE INVENTION

Acute ischemic stroke and other thrombotic conditions represent some of the most prevalent medical problems today. The standard treatment for ischemic stroke is the administration of a thrombolytic agent such as tissue plasminogen activator (tPA). To be effective, the thrombolytic agent should be administered within the first several hours following the onset of symptoms. Administration of a thrombolytic agent, however, increases the risk for intracranial hemorrhage. Thus, there is a need for safer and more effective methods for treating thrombus-associated conditions.

### SUMMARY OF THE INVENTION

Among the various aspects of the present disclosure is the provision of a method for lysing a thrombus in a subject in need thereof. The method comprises administering a plurality of biodegradable nanoparticles having an average diameter less than about 500 nm to the subject, and delivering ultrasound to the subject such that the thrombus is lysed.

Another aspect of the present disclosure provides a method for ablating a mass in a subject in need thereof. The method comprises administering a plurality of biodegradable nanoparticles having an average diameter less than about 500 nm to the subject, and delivering ultrasound to the subject such that the mass is ablated.

Still another aspect of the present disclosure encompasses a method for delivering a therapeutic agent to a tumor in a subject in need thereof. The method comprises administering a plurality of biodegradable nanoparticles comprising the therapeutic agent and having an average diameter less than about 500 nm to the subject, and delivering ultrasound to the subject such that the therapeutic agent is delivered to the tumor.

Other aspects and iterations of the disclosure are described in more detail below.

### BRIEF DESCRIPTION OF THE DRAWINGS

FIG. 1 depicts a graph showing the potentiation of sonothrombolysis (STBL) efficacy by microbubbles (MBs) and/or a nanoemulsion of cyclopentadecanolide with or without tissue plasminogen activator (tPA). This figure also shows that STBL efficacy was potentiated markedly when biodegradable starch nanoparticles (StNPs) (450 nm diameter) were combined with MBs and tPA. STBL was performed with fibrin-rich clots (see Example 2) using pulsed, 1 MHz ultrasound for 15 min. The MBs, tPA, StNPs etc. were suspended in bovine calf serum and introduced at a flow rate of 0.5 mL/min. STBL efficacy is reported as % clot lysis. Abbreviations: US=ultrasound; MBs=microbubbles; CPDL=cyclopentadecanolide; StNPs=starch nanoparticles; tPA=tissue plasminogen activator.

FIG. 2 depicts a graph showing STBL efficacy as a function of starch nanoparticle (StNP) concentration in OD₅₃₀ units. The StNPs had an average diameter of 250 nm. STBL efficacy is reported as % clot lysis. 1 OD₅₃₀ unit is equal to 1.8×10¹⁰ StNPs/mL.

FIG. 3 depicts a graph showing STBL efficacy with starch nanoparticles as a function of ultrasonic intensity at 1 MHz or 3 MHz and varying ultrasonic intensities. STBL efficacy is reported as % clot lysis.

FIG. 4 depicts a graph showing STBL efficacy with or without starch nanoparticles (250 nm diameter) as a function of temperature at 25° C. and 37° C. STBL efficacy is reported as % clot lysis.

FIG. 5 depicts a graph showing STBL efficacy with either microbubbles or starch nanoparticles (250 nm diameter) as a function of thrombin units added to the clot. STBL efficacy is reported as % clot lysis.

### DETAILED DESCRIPTION OF THE INVENTION

Provided herein is a method for sonothrombolysis in which thrombi are lysed or destroyed in a subject by the administration of biodegradable nanoparticles and ultrasound to the subject. Applicants of the present invention discovered that biodegradable nanoparticles (e.g., starch nanoparticles) could be used in combination with ultrasound to lyse thrombi without the use of thrombolytic agents. Moreover, it was discovered that the combination of biodegradable nanoparticles and ultrasound could effectively lyse thrombi that were not freshly formed. Thus, the disclosed method may be used to lyse thrombi in acute ischemic stroke patients after the critical three hour time period.

Also provided are methods for using the biodegradable nanoparticles and ultrasound to ablate tumors, plaques, fibroids, or other entities in subjects, as well as methods for delivering chemotherapeutic agents to tumors in subjects.

### (I) Methods for Lysing Thrombi

One aspect of the present disclosure encompasses a method for lysing a thrombus in a subject in need thereof. The method comprises administering to the subject a plurality of biodegradable nanoparticles having an average diameter of less than about 500 nm, and delivering ultrasound to the subject such that the thrombus is lysed. As used herein, the term thrombus refers to one or more blood clots.

(a) Biodegradable Nanoparticles

The method comprises administering to the subject a plurality of biodegradable nanoparticles. The biodegradable nanoparticles comprise a material chosen from natural polymers, such as polysaccharides and proteins, synthetic biodegradable polymers, variants or derivatives thereof, and combinations thereof.

Non-limiting examples of suitable polysaccharides include starch, amylose, amylopectin, cellulose, arabinoxylan, chitin, chitinosan, pectin, alginate, carageenan, dextrin, gums (e.g., arabic gum, gellan gum, guar gum, locust bean gum, xanthan gum), or combinations thereof. Examples of suitable proteins include but are not limited to serum albumin, egg albumin, casein, collagen, gelatin, soy protein, whey protein, zein, or combinations thereof. Non-limiting examples of suitable synthetic biodegradable polymers include poly-lactide, poly-D-L-glycolide, poly-D-L-lactide-coglycolide, poly-ε-caprolactone, polyalkylenglycol (e.g., polyethyleneglycol), 1,3-propanediol, 1,4-butanediol, polycyanoacrylate, polyalkylcyanoacrylate, polyanhydride, polyorthoester, or combinations thereof. The polysaccharide, protein, or synthetic biodegradable polymer may be derivatized with one or more groups chosen from alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The average diameter of the biodegradable nanoparticles can and will vary. In general, the average diameter of the biodegradable nanoparticles may range from about 1 nm to about 900 nm. In various embodiments, the average diameter of the biodegradable nanoparticles may be about 850, 800, 750, 700, 650, 600, 550, 500, 450, 400, 350, 300, 250, 200, 150, 100, 50, 20, or 10 nm. In exemplary embodiments, the average diameter of the biodegradable nanoparticles may be less than about 500 nm.

In some embodiments, about 60% of the biodegradable nanoparticles have an average diameter of 450, 400, 350, 300, 250, 200, 150, 100, or 50 nm. In other embodiments, about 70% of the biodegradable nanoparticles have an average diameter 450, 400, 350, 300, 250, 200, 150, 100, or 50 nm. In still other embodiments, about 80% of the biodegradable nanoparticles have an average diameter of 450, 400, 350, 300, 250, 200, 150, 100, or 50 nm. In further embodiments, about 90% of the biodegradable nanoparticles have an average diameter of 450, 400, 350, 300, 250, 200, 150, 100, or 50 nm.

In exemplary embodiments, the biodegradable nanoparticles are starch nanoparticles. The biodegradable starch nanoparticles may be prepared from corn starch, wheat starch, pea starch, bean starch, tapioca starch, potato starch, cassava starch, rice starch, or another vegetable starch. The starch particles may be prepared using methods known in the art. Alternatively, the biodegradable starch nanoparticles may be purchased from a commercial entity. In some embodiments, the biodegradable starch nanoparticles may have diameters that range from about 50-100 nm, 100-150 nm, 150-200 nm, 200-250 nm, 250-300 nm, 300-350 nm, 350-400 nm, 400-450 nm, or 450-500 nm. In exemplary embodiments, the biodegradable starch nanoparticles may have an average diameter of about 100 nm, or about 200 nm.

(b) Routes of Administration

The route of administration of the plurality of biodegradable nanoparticles can and will vary depending upon the location of the thrombus. In some embodiments, the plurality of biodegradable nanoparticles may be administered via an artery (i.e., intra-arterially). For example, the plurality of biodegradable nanoparticles may be introduced into a carotid artery, a subclavian artery, a femoral artery, a pulmonary artery, an aortic artery, a cerebral artery, a coronary artery, a systemic artery, or another artery. In other embodiments, the plurality of biodegradable nanoparticles may be administered via a vein (i.e., intravenously). For example, the plurality of biodegradable nanoparticles may be introduced into a femoral vein, a brachial vein, a pulmonary vein, a breast vein, a cerebral vein, a brain sinus vein, a renal vein, a portal vein, a jugular vein, or another vein.

The concentration of biodegradable nanoparticles administered to the subject can and will vary depending on the diameter of the nanoparticles as well as the size, density, and age of the thrombus. In various embodiments, the concentration of biodegradable nanoparticles administered to the subject may range from about 10⁷ to about 10⁸ nanoparticles per mL, from about 10⁸ to about 10⁹ nanoparticles per mL, from about a 10⁹ to about 10¹⁰ nanoparticles per mL, from about 10¹⁰ to about 10¹¹ nanoparticles per mL, from about 10¹¹ to about 10¹² nanoparticles per mL, from about 10¹² to about 10¹³ nanoparticles per mL, or more than about 10¹³ nanoparticles per mL. The nanoparticles generally will be administered in a suitable vehicle, such as sterile saline, sterile phosphate saline, a sterile buffer solution, blood plasma, or whole blood.

Typically the subject will be a vertebrate. In some embodiments, the subject may be a mammal. Non-limiting examples of mammals include humans, a non-human primates, cats, dogs, cows/cattle, pigs, sheep, horses, rodents, rabbits, zoo animals, research animals, and the like. In other embodiments, the subject may be a bird, a fowl, an aquatic animal, or a frog. In exemplary embodiments, the subject is a human.

(c) Ultrasound

The method further comprises delivering ultrasound to the subject such that the combination of ultrasound and the nanoparticles lyse the thrombus. Without being bound to any particular theory, it is proposed that the ultrasound-mediated projection of the small sized nanoparticles destructs or disintegrates the thrombus. In general, the ultrasound is delivered to the region of the subject in which the thrombus is located.

The intensity and frequency of the ultrasound delivered to the subject can and will vary depending upon a variety of factors including the location, size, and age of the thrombus. In various embodiments, the frequency of the ultrasound delivered to the subject may range from about 0.1-0.3 MHz, from about 0.3-1 MHz, from about 1-3 MHz, from about 4-10 MHz, from about 10-30 MHz, from about 30-100 MHz, or from about 100-300 MHz. The intensity of the ultrasound may also vary. In certain embodiments, the intensity of the ultrasound delivered to the subject may range from about 0.005-0.1 W/cm², from about 0.1-0.3 W/cm², from about 0.3-1.0 W/cm², about 1-3 W/cm², about 3-10 W/cm², from about 10-30 W/cm², from about 100-300 W/cm², or from about 300-1000 W/cm².

Ultrasound of different frequencies and/or intensities may be delivered simultaneously or sequentially to the subject. Delivery of the ultrasound may be continuous or pulsed (i.e., discontinuous). The duration of the ultrasound delivery also may vary. For example, the ultrasound may be delivered for less than one minute, from about 1-3 minutes, from about 3-10 minutes, from about 10-30 minutes, from about 30-100 minutes, from about 1-3 hours, from about 3-10 hours, or from about 10-30 hours.

A variety of ultrasound delivery devices may be used to deliver the ultrasound to the subject. Suitable devices are well known in the art. The ultrasound may be delivered via a transducer probe. The transducer probe may be applied externally or it may be implanted in the subject. Alternatively, the ultrasound may be delivered via catheter. For example, an ultrasound catheter may be inserted into an artery for intra-arterial administration of ultrasound.

(d) Thrombus Lysis

The method comprises administering a plurality of biodegradable nanoparticles and delivering ultrasound to a thrombus in a subject. The thrombus may be a mural thrombus that reduces blood flow through the blood vessel, or the thrombus may be an occlusion thrombus that blocks blood flow through the blood vessel. The thrombus may be due to venous thrombosis (i.e., superficial or deep venous thrombosis), portal vein thrombosis, renal vein thrombosis, jugular vein thrombosis, hepatic vein thrombosis, cerebral venous sinus thrombosis, pulmonary embolism, arterial thrombosis, atherothrombosis, ischemic stroke, thombotic stroke, embolic stroke, hemorrhagic stroke, brain ischemia, cerebral infarction, myocardial infarction, ischemic heart disease, disturbed blood flow (e.g., atrial fibrillation, heart failure, cardiac arrhythmias), vavlulitis, splenic infarction, limb infarction, aneurysms, ischemic colitis, endothelial cell injuries, hypercoagulability conditions, and the like.

The age of the thrombus may vary. In some embodiments, the thrombus may fresh, e.g., it may have been formed in the past 0-3 hours, in the past 3-6 hours, in the past 6-12 hours, or in the past 12-24 hours. In other embodiments, the thrombus may have formed in the past 1-2 days, in the past 2-4 days, in the past 4-7 days, in the past 1-2 weeks, in the past 2-4 weeks, in the past 1-2 months, in the past 2-6 months, longer than 6 months ago, or even having formed years ago in one region and then being relocated to a critical location elsewhere in the body. The age of the thrombus may be estimated based upon the onset of symptoms presented by the subject. Alternatively, the age of the thrombus may be estimated using standard histological techniques known in the art.

Upon administration of the biodegradable nanoparticles and delivery of ultrasound to the subject, the thrombus is lysed or destroyed. The lysis of the thrombus may be partial or essentially complete. For example, the thrombus may be reduced in size such that blood flow increases through the vessel. Alternatively, the thrombus may be substantially eliminated such that blood flow is restored to pre-thrombus levels. In some embodiments, the thrombus may be reduced in size (or volume) by at least about 5, 10, 15, 20, 25, 30, 35, 40, 45, 50, 55, 60, 65, 70, 75, 80, 85, 90, 95, or 99%. In other embodiments, blood flow through the blood vessel comprising the thrombus may be increased by at least about 5, 10, 15, 20, 25, 30, 35, 40, 45, 50, 55, 60, 65, 70, 75, 80, 85, 90, 95, or 99%.

(e) Optional Thrombolytic Agent

In some embodiments, the biodegradable nanoparticles may further comprise a thrombolytic agent. Suitable thrombolytic agents include tissue plasminogen activator (tPA), recombinant tPA (e.g., alteplase, reteplase, tenecteplase, desmoteplase), anistreplase, streptokinase, and urokinase. In some embodiments, the thrombolytic agent may be absorbed by the biodegradable nanoparticle. In other embodiments, the thrombolytic agent may be adsorbed to the surface of the biodegradable nanoparticles (i.e., via noncovalent interactions). In still other embodiments, the thrombolytic agent may be covalently attached to the biodegradable nanoparticles. The covalent attachment may be direct between the thrombolytic agent and the nanoparticle, or the covalent linkage may be indirect via a linker molecule. Suitable linker molecules are well known in the art.

In other embodiments, the method further comprises administering a thrombolytic agent to the subject. Typically, the thrombolytic agent will be administered intravenously. The thrombolytic agent may be administered prior to, concurrently with, or after administration of the biodegradable nanoparticles.

(f) Optional Microbubbles or Nanobubbles

In some embodiments, the method may further comprise administering a plurality of microbubbles and/or a plurality of nanobubbles. In general, microbubbles are micrometer sized (i.e., 1-20 μm in diameter) bubbles comprising a gaseous interior and a lipid or protein outer shell. Nanobubbles are nanometer sized (10-999 nm in diameter) variants of microbubbles. The microbubbles or nanobubbles may cavitate in response to the ultrasound such that the resultant microstreaming may provide additional energy to propel the biodegradable nanoparticles into the thrombus.

Exemplary microbubbles comprise an outer protein/saccharide shell and a perfluorocarbon interior. These exemplary microbubbles are described in U.S. Publication No. 2011/0044903, which is hereby incorporated by reference in its entirety. Nanobubbles may be produced using the same formulation but using different ultrasound settings to produce the nanometer sized bubbles.

The microbubbles and/or nanobubbles may be administered prior to, concurrent with, or after the biodegradable nanoparticles. Typically, the microbubbles and/or nanobubbles will be administered via the same route as the biodegradable nanoparticles. The dose of microbubbles and/or nanobubbles can and will vary depending upon the size and composition of the microbubbles and/or nanobubbles, as well as the size, location, and age of the thrombus.

### (II) Method for Ablating a Mass in a Subject

Another aspect of the disclosure provides a method for ablating a mass in a subject. The method comprises administering a plurality of biodegradable nanoparticles having an average diameter less than about 500 nm to the subject, and delivering ultrasound to the subject such that the mass is ablated.

The identity of the mass can and will vary. In some embodiments, the mass may be a tissue mass or a collection of cells. For example, the mass may be a tumor (or neoplasm), which may be malignant or benign. Malignant tumors may be associated with a variety of different cancers (e.g., lung, brain, eye, bone, esophagus, stomach, GI, colon, liver, bladder, breast, cervical, uterine, ovarian, pancreatic, gallbladder kidney, thyroid, thymus, prostate, penile, etc.) In other embodiments, the mass may be a fibroid (e.g., a uterine fibroid), a polyp, a keloid, a granuloma, a cyst, a thrombus, or an atherosclerotic plaque. In alternate embodiments, the mass may be a calcium deposit, a renal calculus, a gallstone, or a cataract. In still further embodiments, the mass may be a collection of bacteria, a biofilm, fungi, or microbes.

Biodegradable nanoparticles suitable for use in the method are detailed above in section (I)(a). In exemplary embodiments, the biodegradable nanoparticles are starch nanoparticles. In some embodiments, the biodegradable starch nanoparticles may have diameters that range from about 50-100 nm, 100-150 nm, 150-200 nm, 200-250 nm, 250-300 nm, 300-350 nm, 350-400 nm, 400-450 nm, or 450-500 nm. In exemplary embodiments, the biodegradable starch nanoparticles may have an average diameter of about 100 nm, or about 200 nm.

The biodegradable nanoparticles may be administered to the subject by a variety of different routes. In general, the route of administration will depend upon the type and/or location of the mass. The plurality of biodegradable nanoparticles may be administered orally (e.g., enterally, sublingually, sublabially, buccally, rectally), dermally, via the respiratory tract, ophthalically, otologically, nasally, urogenitally, parenterally (e.g., intradermally, subcutaneously, percutaneously, transdermally, intravenously, intra-arterially, intracardially, intramuscularly, intraperitoneally, intraosseously, intrathecally, intraventricularly, intracerebrally, epidurally, intracavernously, intravitreally, intra-articularly, transsclerally) or combinations thereof.

Typically the subject will be a vertebrate, as detailed above. In exemplary embodiments, the subject is a mammal, for example, a human.

The method further comprises delivering ultrasound (which is detailed above in section (I)(c) to the subject such that the mass is ablated. In some embodiments, the mass is reduced in size (or volume) by at least about 5, 10, 15, 20, 25, 30, 35, 40, 45, 50, 55, 60, 65, 70, 75, 80, 85, 90, 95, or 99%.

In some embodiments, the method may further comprise administering a plurality of microbubbles and/or nanobubbles, as detailed above in section (I)(f).

### (III) Method for Delivering a Therapeutic Agent to a Tumor in a Subject

Still another aspect of the disclosure encompasses a method for delivering a therapeutic agent to a tumor in a subject. The therapeutic agent may be a chemotherapeutic agent or a radiotherapeutic agent. The method comprises administering a plurality of biodegradable nanoparticles comprising the therapeutic agent and having an average diameter less than about 500 nm to the subject, and delivering ultrasound to the subject such that the therapeutic agent is delivered to the tumor. Thus, the method comprises delivering of biodegradable nanoparticles comprising the therapeutic agent to the tumor via sonophoresis such that the chemotherapeutic agent or radiotherapeutic agent is able interact with the tumor.

The method may be used to deliver a variety of therapeutic agents to a variety of tumors. Non-limiting examples of tumors are presented above in section (II). The therapeutic agent is delivered to the tumor via the degradable nanoparticles as detailed above in section (I)(a). Stated another way, the biodegradable nanoparticles further comprise a therapeutic agent. The therapeutic agent may be a chemotherapeutic agent or a radiotherapeutic agent. The type of therapeutic agent can and will vary depending on the type of tumor and/or progression (i.e., stage) of the tumor. The therapeutic agent may be a cytotoxic agent that affects rapidly dividing cells in general, or it may be a targeted therapeutic agent that affects the deregulated proteins of cancer cells. In general, the therapeutic agent may be an alkylating agent, an anti-metabolite, an anti-tumor antibiotic, an anti-cytoskeletal agent, a topoisomerase inhibitor, an anti-hormonal agent, a targeted therapeutic agent, a radioisotope, or a combination thereof.

Non-limiting examples of suitable alkylating agents include altretamine, benzodopa, busulfan, carboplatin, carboquone, carmustine (BCNU), chlorambucil, chlornaphazine, cholophosphamide, chlorozotocin, cisplatin, cyclosphosphamide, dacarbazine (DTIC), estramustine, fotemustine, ifosfamide, improsulfan, lomustine (CCNU), mechlorethamine, mechlorethamine oxide hydrochloride, melphalan, meturedopa, nimustine, novembichin, phenesterine, piposulfan, prednimustine, ranimustine; temozolomide, thiotepa, triethylenemelamine, trietylenephosphoramide, triethylenethiophosphaoramide, trimethylolomelamine, trofosfamide, uracil mustard and uredopa. Suitable anti-metabolites include, but are not limited to aminopterin, ancitabine, azacitidine, 6-azauridine, capecitabine, carmofur (1-hexylcarbomoyl-5-fluorouracil), cladribine, cytarabine or cytosine arabinoside (Ara-C), dideoxyuridine, denopterin, doxifluridine, enocitabine, floxuridine, fludarabine, 5-fluorouracil, gemcetabine, hydroxyurea, leucovorin (folinic acid), 6-mercaptopurine, methotrexate, pemetrexed, pteropterin, thiamiprine, trimetrexate, and thioguanine. Non-limiting examples of suitable anti-tumor antibiotics include aclacinomysin, actinomycins, adriamycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mithramycin, mycophenolic acid, nogalamycin, olivomycins, peplomycin, plicamycin, potfiromycin, puromycin, quelamycin, rodorubicin, streptonigrin, streptozocin, tubercidin, valrubicin, ubenimex, zinostatin, and zorubicin. Non-limiting examples of suitable anti-cytoskeletal agents include colchicines, docetaxel, macromycin, paclitaxel, vinblastine, vincristine, vindesine, and vinorelbine. Suitable topoisomerase inhibitors include, but are not limited to, amsacrine, etoposide (VP-16), irinotecan, mitoxantrone, RFS 2000, teniposide, and topotecan. Non-limiting examples of suitable anti-hormonal agents such as aminoglutethimide, aromatase inhibiting 4(5)-imidazoles, bicalutamide, finasteride, flutamide, goserelin, 4-hydroxytamoxifen, keoxifene, leuprolide, LY117018, mitotane, nilutamide, onapristone, raloxifene, tamoxifen, toremifene, and trilostane. Examples of targeted therapeutic agents include, without limit, monoclonal antibodies such as alemtuzumab, epratuzumab, gemtuzumab, ibritumomab tiuxetan, rituximab, tositumomab, and trastuzumab; protein kinase inhibitors such as bevacizumab, cetuximab, dasatinib, erlotinib, gefitinib, imatinib, lapatinib, mubritinib, nilotinib, panitumumab, pazopanib, sorafenib, sunitinib, and vandetanib; angiogeneisis inhibitors such as angiostatin, endostatin, bevacizumab, genistein, interferon alpha, interleukin-2, interleukin-12, pazopanib, pegaptanib, ranibizumab, rapamycin, thalidomide; and growth inhibitory polypeptides such as erythropoietin, interleukins (e.g., IL-1, IL-2, IL-3, IL-6), leukemia inhibitory factor, interferons, thrombopoietin, TNF-α, CD30 ligand, 4-1BB ligand, and Apo-1 ligand. Non-limiting examples of suitable radioisotopes include Iodine-131, Iodine-125, Iodine-124, Lutecium-177, Phosphorous-132, Rhenium-186, Strontium-89, Yttrium-90, Iridium-192, and Samarium-153. Also included are pharmaceutically acceptable salts, acids, or derivatives of any of the above listed agents.

In some embodiments, the therapeutic agent may be absorbed by the biodegradable nanoparticle. In other embodiments, the therapeutic agent may be adsorbed to the surface of the biodegradable nanoparticles (i.e., via noncovalent interactions). In still other embodiments, the therapeutic agent may be covalently attached to the biodegradable nanoparticles. The covalent attachment may be direct between the therapeutic agent and the nanoparticle, or the covalent linkage may be indirect via a linker molecule. Suitable linker molecules are well known in the art.

The biodegradable nanoparticles comprising the therapeutic agent may be administered to the subject by a variety of routes depending on the location of the tumor. In some embodiments, the route of administration may be intratumoral. In other embodiments, the route of administration may be parenteral, oral, etc., as described above in section (II). As mentioned above, typically the subject will be a vertebrate. In exemplary embodiments, the subject is a mammal. An exemplary mammal is a human.

In some embodiments, the method may further comprise administering a plurality of microbubbles and/or nanobubbles as detailed above in section (I)(f).

### Definitions

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The terms “ablate,” “ablating,” or “ablation,” as used herein, refer to the destruction of a tissue, a cellular mass, or a non-cellular mass by an erosive process.

The term “biodegradable” refers to organic material originating from living organisms or artificial material that is sufficiently similar to said organic material to be utilized by cellular enzymes and/or microorganisms. Biodegradable material may be fully degradable or partially degradable by cellular enzymes and/or microorganisms. Partial degradation may range from about 0.1% to about 99.9%.

As used herein, the terms “lyse,” “lysing,” or “lysis” refer to the dissolution, disintegration, or destruction of a thrombus.

Having described the invention in detail, it will be apparent that modifications and variations are possible without departing from the scope of the invention defined in the appended claims.

### EXAMPLES

The following examples illustrate various iterations of the invention.

### Example 1

### Sonothrombolysis with Iron and Gold Nanoparticles in Combination with Tissue Plasminogen Activator and/or Microbubbles

Traditional sonothrombolysis (STBL) with tissue plasminogen activator (tPA) and/or microbubbles (MBs) is markedly less effective on thrombi that are more aged and/or enriched with fibrin and/or platelets and/or other cells or cellular debris. Suspending the MBs and/or diluting the tPA in a nanoemulsion of cyclopentadecanolide, a tissue permeabilizing agent, improved efficiency, but a still more effective approach was desired.

It was hypothesized that the microstreaming (jets of sound and liquid) produced by microbubbles during sonothrombolysis would propel nanoparticles into the thrombus such that the nanoparticles (NPs) would function as an abrasive to help lyse/erode the clot (e.g. a nanoscale version of sand blasting). Gold (70-130 nm in diameter) or iron (100 nm in diameter) nanoparticles were combined with 3 μm diameter MBs and pulsed ultrasound STBL of the more rigid and aged clots (formed with 60 U added thrombin, aged for 24 h at 37° C., and then for 24 h at 5° C.) was performed. It was found that STBL efficacy with the combination of the gold or iron NPs and MBs was enhanced more than two-fold over that achieved with MBs alone, using the same ultrasound parameters.

### Example 2

### STBL with Starch Nanoparticles in Combination with Tissue Plasminogen Activator, Microbubbles, and/or Cyclopentadecanolide

Due to toxicity concerns with using either gold or iron NPs, a nontoxic, biodegradable NP for sonothrombolysis was investigated. Starch nanoparticles (StNPs) are biodegradable and nontoxic. Thus, rather than gold or iron NPs, StNPs with a mean diameter of approximately 450 nm were used in the STBL experiments.

The clots used in these experiments were formed by mixing pooled rabbit plasma with rabbit blood cells (final volume equal to 2 mL; the volume of whole rabbit blood from which the cells were obtained), incubating this mixture for 24 h at 37° C. and then curing the formed clots for 24 h at 5° C. Pieces of the clots (12-15 mg) were subjected to in vitro sonothrombolysis (STBL) using pulsed, 1 MHz ultrasound (pulse duration 2 ms, pulse repeat frequency 100 Hz, duty factor 20%) for 15 min in the presence of various combinations of 3 μm MBs, 0.06% w/V nanoemulsion of cyclopentadecanolide (CP DL), 0.1 mg/ml tPA, and/or 450 nm starch nanoparticles (StNPs). The MBs, tPA, StNPs, etc. were suspended in bovine calf serum and then flowed past the suspended clot at a rate of 0.5 mL/min. The StNPs were diluted to have an Optical Density reading at 530 nm (OD₅₃₀) of 1.0. STBL with ultrasound alone was used as a control. STBL efficacy was measured as the percent of the initial clot mass that was lost from the clot during the 15 min treatment time.

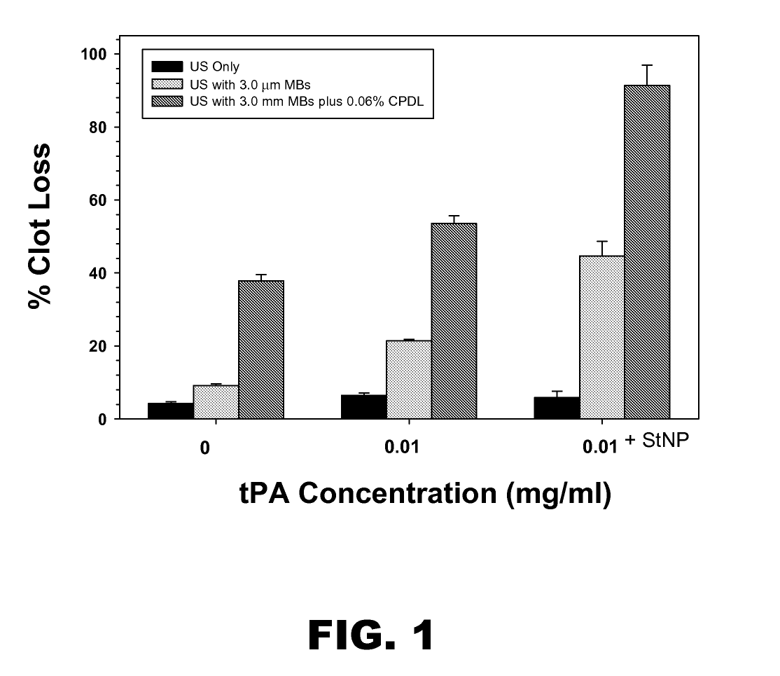

The results are shown in FIG. 1. Using MBs or tPA improved STBL efficacy, and better results were obtained when MBs and tPA were combined, as reported previously. Adding CPDL improved STBL efficacy markedly, and a further improvement in STBL efficacy was achieved when StNPs were combined with tPA, tPA and MBs, or tPA, MPs and CPDL. The most effective STBL was achieved when the StNPs were added to a combination of tPA, MBs, and CPDL, which resulted in a clot mass loss of nearly 90% of the original clot mass.

### Example 3

### STBL with Starch Nanoparticles

It was hypothesized that particle size may impact the ability of the MBs to accelerate the StNP to a high projectile velocity. To explore whether StNPs with a smaller diameter would result in increased efficacy, StNPs with an average diameter of 250 nm (as measured using a NanoSight system (NanoSight Ltd., Minton Park, London Road, Amesbury Wiltshire, SP4 7RT, United Kingdom) were tested.

The clots used in these experiments were fibrin-rich, formed by adding 60 U of thrombin to a mixture of pooled rabbit plasma with rabbit blood cells (final volume equal to 2 mL; the volume of whole rabbit blood from which the cells were obtained), incubating this mixture for 24 h at 37° C. and then curing the formed clots for 24 h at 5° C. Pieces of the clots (12-15 mg) were subjected to in vitro STBL using pulsed, 1 MHz ultrasound (pulse duration 2 ms, pulse repeat frequency 100 Hz, duty factor 20%) for 15 min. The StNPs were suspended in bovine calf serum and then flowed past the suspended clot at a rate of 0.5 mL/min. Afterwards, the clot was removed from the chamber, adherent serum was wicked away from the clot, and the clot piece was weighed again with an analytical balance. The fractional mass loss from the clot was determined, and the % clot loss was used as a quantitative measure of STBL efficacy.

Surprisingly, these experiments revealed that STNPs alone, without MPs or tPA or CPDL, were capable of promoting STBL.

### Example 4

### STBL Efficacy as a Function of StNP Concentration

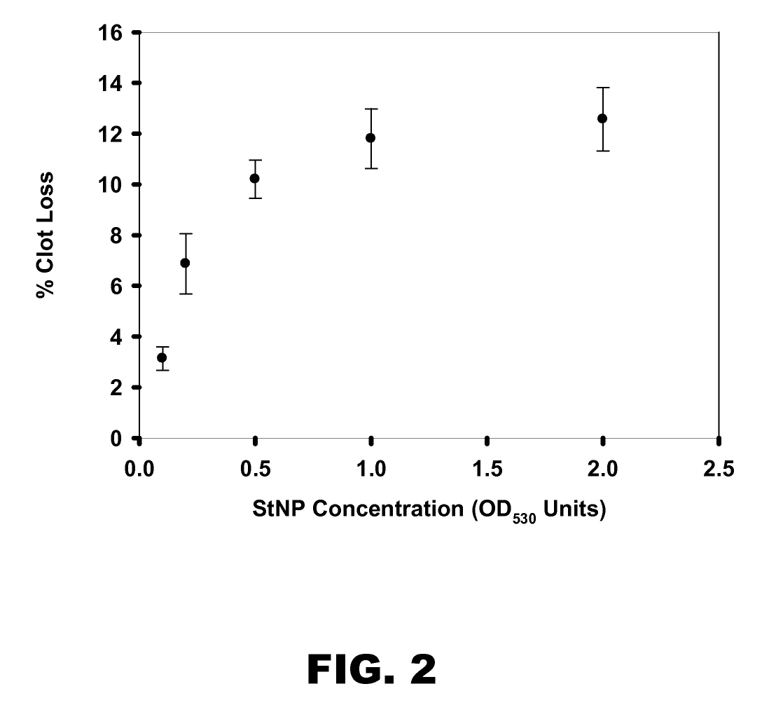

Next, STBL efficacy as a function of StNP concentration was determined. These STBL experiments were performed using StNPs and ultrasound, with no other additives (no MBs, tPA, etc.). The concentration of StNPs was varied from 0.1 to 2.0 OD₅₃₀ units (i.e., 0.18×10¹⁰ StNPs/mL to 3.6×10¹⁰ StNPs/mL). Measurements using the Nanosight Instrument at the National Center for Toxicological Research (NCTR) showed that 1 OD₅₃₀ unit is equal to 1.8×10¹⁰ StNPs/mL. STBL was performed as detailed in Example 3. FIG. 2 shows the variation of STBL efficacy as a function of StNP concentration using pulsed 1 MHz ultrasound with a spatial average temporal average ultrasonic intensity of 0.1 W/cm². Maximal STBL efficacy was achieved with a StNP concentration of 1 OD₅₃₀ unit, or 1.8×10¹⁰ StNPs/mL.

### Example 5

### STBL Efficacy as a Function of Ultrasonic Intensity

STBL efficacy as a function of ultrasonic intensity was next determined. The concentration of StNBs (250 nm diameter) was 1 OD₅₃₀ unit (1.8×10¹⁰ StNPs/mL). STBL was performed as detailed in Example 3. The StNPs were suspended in bovine calf serum and then flowed past the suspended clot at a rate of 0.5 mL/min

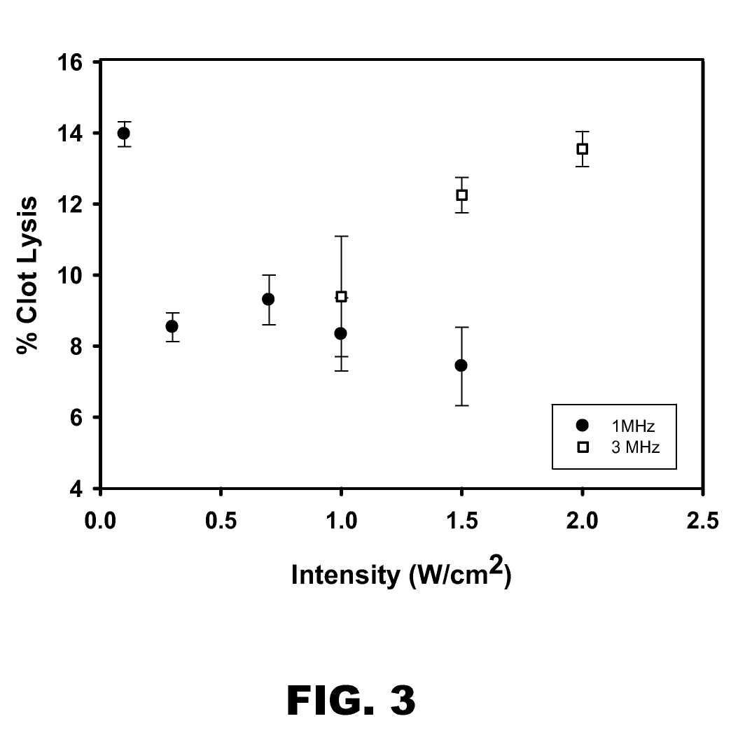

FIG. 3 shows the variation in STBL efficacy as the spatial average temporal average pulsed ultrasonic intensity was varied and all other parameters were held constant. Maximal STBL efficacy at 1 MHz was achieved with an intensity of 0.1 W/cm². Increasing the intensity to 1 MHz resulted in a sharp decrease in STBL efficacy. Maximal STBL at 3 MHz was achieved using an intensity of 2 W/cm². A higher intensity was not attempted as the transducer was not rated for being used with a higher intensity. However, still more efficient STBL using acoustic intensities greater than 2 W/cm² may be achievable.

### Example 6

### STBL Efficacy as a Function of Temperature

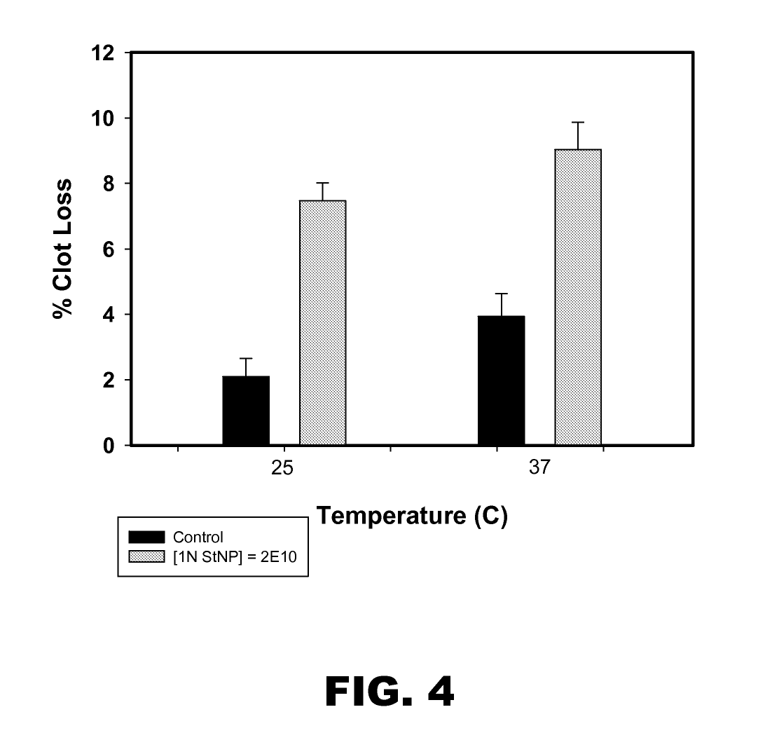

STBL efficacy as a function of temperature was also examined. In these experiments, StNPs (250 nm, 1 OD₅₃₀ unit) were suspended in PBS. STBL was performed at 25° C. or 37° C. The control experiment was STBL with ultrasound only and no StNPs. The results are presented in FIG. 4. There was a slight increase in STBL efficacy when the temperature was increased from 25° C. to 37° C. in the absence or presence of StNPs, which could be attributed to the increased activity of nascent tPA and plasmin present within the clot itself.

### Example 7

### STBL Efficacy as a Function of Clot Fibrin Content and Complexity

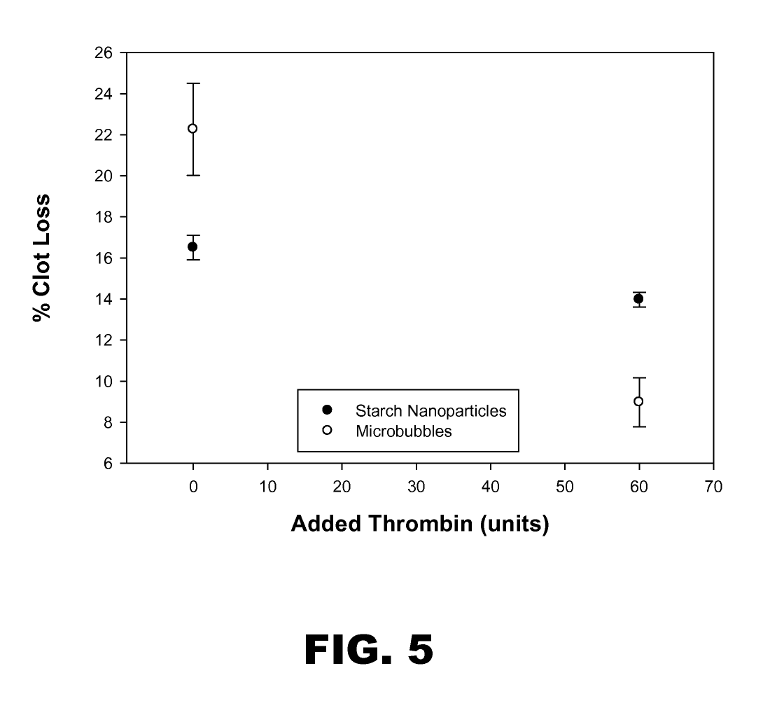

Lastly, the effect of clot fibrin content and complexity on STBL efficacy with MBs or StNPs was determined. STBL efficacy with MBs (3 μm, 1.128×10⁸ MB/mL) or StNPs (250 nm; 1.8×10¹⁰ StNB/mL) was tested with clots that had different levels of fibrin content. The fibrin content/complexity was adjusted by adding thrombin to the blood at the time of clot formation. Data are shown in FIG. 5. Overall, the STBL efficacy with MBs or StNPs was comparable. However, STBL with MBs was statistically more effective for clots with lower fibrin content/complexity while STBL with StNPs was more effective for clots with higher fibrin content/complexity.

1. A method for lysing a thrombus in a subject in need thereof, the method comprising administering a plurality of biodegradable nanoparticles having an average diameter less than about 500 nm to the subject; and delivering ultrasound to the subject such that the thrombus is lysed.

2. The method of claim 1, wherein the plurality of biodegradable nanoparticles comprises a material chosen from a polysaccharide, a protein, a synthetic biodegradable polymer, or a combination thereof, and wherein the polysaccharide is chosen from starch, cellulose, pectin, chitosan, or a combination thereof; the protein is chosen from gelatin, collagen, albumin, or a combination thereof; and the synthetic biodegradable polymer is chosen from poly-lactide, poly-D-L-glycolide, poly-D-L-lactide-coglycolide, poly-ε-caprolactone, polyalkylenglycol, 1,3-propanediol, 1,4-butanediol, polycyanoacrylate, polyalkylcyanoacrylate, polyanhydride, polyorthoester, or a combination thereof.

3. (canceled)

4. The method of claim 1, wherein the plurality of biodegradable nanoparticles comprises starch nanoparticles.

5. The method of claim 4, wherein the average diameter of the starch nanoparticles is about 200 nm.

6. The method of claim 4, wherein the average diameter of the starch nanoparticles is about 100 nm.

7. The method of claim 1, wherein the plurality of biodegradable nanoparticles is administered intra-arterially or intravenously.

8. The method of claim 1, wherein the thrombus is due to venous thrombosis, arterial thrombosis, stroke, myocardial infarction, endothelial cell injury, or disturbed blood flow.

9. The method of claim 1, wherein the plurality of biodegradable nanoparticles further comprise a thrombolytic agent chosen from tissue plasminogen activator, recombinant tissue plasminogen activator, anistreplase, streptokinase, and urokinase.

10. The method of claim 1, further comprising administering a thrombolytic agent chosen from tissue plasminogen activator, recombinant tissue plasminogen activator, anistreplase, streptokinase, and urokinase to the subject.

11. The method of claim 1, wherein the subject is a human.

12. A method for ablating a mass in a subject in need thereof, the method comprising administering a plurality of biodegradable nanoparticles having an average diameter less than about 500 nm to the subject; and delivering ultrasound to the subject such that the mass is ablated.

13. The method of claim 12, wherein the mass is a tumor, an atherosclerotic plaque, a uterine fibroid, a renal calculus, a gallstone, or a polyp.

14. The method of claim 12, wherein the plurality of biodegradable nanoparticles comprises a material chosen from a polysaccharide, a protein, a synthetic biodegradable polymer, or a combination thereof, and wherein the polysaccharide is chosen from starch, cellulose, pectin, chitosan, or a combination thereof; the protein is chosen from gelatin, collagen, albumin, or a combination thereof; and the synthetic biodegradable polymer is chosen from poly-lactide, poly-D-L-glycolide, poly-D-L-lactide-coglycolide, poly-ε-caprolactone, polyalkylenglycol, 1,3-propanediol, 1,4-butanediol, polycyanoacrylate, polyalkylcyanoacrylate, polyanhydride, polyorthoester, or a combination thereof.

15. (canceled)

16. The method of claim 12, wherein the plurality of biodegradable nanoparticles comprises starch nanoparticles.

17. The method of claim 16, wherein the average diameter of the starch nanoparticles is about 200 nm.

18. (canceled)

19. A method for delivering a therapeutic agent to a tumor in a subject in need thereof, the method comprising administering a plurality of biodegradable nanoparticles comprising the therapeutic agent and having an average diameter less than about 500 nm to the subject; and delivering ultrasound to the subject such that the therapeutic agent is delivered to the tumor.

20. The method of claim 19, wherein the therapeutic agent is chosen from alkylating agent, an anti-metabolite, an anti-tumor antibiotic, an anti-cytoskeletal agent, a topoisomerase inhibitor, an anti-hormonal agent, a targeted therapeutic agent, a radioisotope, or combinations thereof.

21. The method of claim 19, wherein the plurality of biodegradable nanoparticles comprises a material chosen from a polysaccharide, a protein, a synthetic biodegradable polymer, or a combination thereof, and wherein the polysaccharide is chosen from starch, cellulose, pectin, chitosan, or a combination thereof; the protein is chosen from gelatin, collagen, albumin, or a combination thereof; and the synthetic biodegradable polymer is chosen from poly-lactide, poly-D-L-glycolide, poly-D-L-lactide-coglycolide, poly-ε-caprolactone, polyalkylenglycol, 1,3-propanediol, 1,4-butanediol, polycyanoacrylate, polyalkylcyanoacrylate, polyanhydride, polyorthoester, or a combination thereof.

22. (canceled)

23. The method of claim 19, wherein the plurality of biodegradable nanoparticles comprises starch nanoparticles.

24. The method of claim 23, wherein the average diameter of the starch nanoparticles is about 200 nm.

25. (canceled)
